(19) United States
(12) Patent Application Publication
(10) Pub. No.: US 2013/0137927 A1
(43) Pub. Date: May 30, 2013

(54) PROPULSION ASSEMBLY FOR ENDOSCOPE AND DRIVING METHOD

(71) Applicant: FUJIFILM CORPORATION, Tokyo (JP)

(72) Inventors: Takayuki NAKAMURA, Ashigarakami-gun (JP); Tsuyoshi ASHIDA, Ashigarakami-gun (JP); Naoyuki MORITA, Ashigarakami-gun (JP); Nobuyuki TORISAWA, Ashigarakami-gun (JP); Takumi DEJIMA, Ashigarakami-gun (JP)

(73) Assignee: FUJIFILM CORPORATION, Tokyo (JP)

(21) Appl. No.: 13/688,477

(22) Filed: Nov. 29, 2012

(30) Foreign Application Priority Data
Nov. 30, 2011 (JP) 2011-261518

(51) Int. Cl. A61B 1/00

(52) U.S. Cl. CPC A61B 1/00133
USPC 600/114

(57) ABSTRACT

A propulsion assembly includes a support sleeve for mounting on a tip device of an endoscope. An endless track device is supported on the support sleeve in an endlessly movable manner, for contacting a wall of a body cavity, for propulsion of the tip device relative to the body cavity. A drive sleeve drives the endless track device. First and second torque wire devices have proximal and distal end portions, the proximal end portion being rotated by a motor, the distal end portion actuating the drive sleeve. Plural helical windings of a first group constitute the first wire device, and are so wound as to increase tightness thereof upon moving the endoscope in a distal direction. Plural helical windings of a second group constitute the second wire device, and are so wound as to increase tightness thereof upon moving the endoscope in a proximal direction.

### BACKGROUND OF THE INVENTION

1. Field of the Invention

The present invention relates to a propulsion assembly for an endoscope and a driving method. More particularly, the present invention relates to a propulsion assembly for an endoscope and a driving method, in which operability for movement of the endoscope can be equal between proximal and distal directions of the movement.

2. Description Related to the Prior Art

An endoscope is a well-known medical device for diagnosis and treatment. The endoscope includes an elongated tube and a tip device. A CCD image sensor is incorporated in the tip device. The elongated tube with the tip device is entered in a body cavity of a body of a patient. An image is created by the CCD image sensor, and displayed on a display panel. An object in the body cavity is imaged and observed by a doctor or operator.

U.S. Pat. Nos. 6,971,990 and 7,736,300 (corresponding to JP-A 2009-513250) and U.S.P. Ser. No. 2005/272,976 (corresponding to JP-A 2005-253892) discloses a propulsion assembly, in which an endless track device is turned around to move the endoscope in a distal direction as an assist function, so as to enter the endoscope in a body cavity even in a very tortuous form, such as a large intestine. The propulsion assembly has a torque wire device of a flexible property for transmitting torque to drive the endless track device. The torque wire device, when rotated in a first direction, causes the endoscope to move in the distal direction, and when rotated in a second direction, causes the endoscope to move in the proximal direction. Examples of the torque wire device are disclosed in U.S.P. Ser. No. 2005/272,976 and JP-A 2001-079007, for example, a device constituted by a plurality of helical windings.

The torque wire device is constituted by combining a plurality of the helical windings in a helical form. According to rotation of the torque wire device in a winding direction and an unwinding direction of the helical windings, that of the torque wire device is lower in the unwinding direction than that of the torque wire device in the winding direction, so that torque applied to the torque wire device in the unwinding direction is difficult to transmit from end to end. The endoscope does not move equally between the proximal and distal directions even when the torque wire device in the propulsion assembly is rotated in the winding direction and the unwinding direction in an equal manner between the directions. A problem occurs in a considerable difference in the operability of the propulsion assembly between the proximal and distal directions of the movement of the endoscope.

### SUMMARY OF THE INVENTION

In view of the foregoing problems, an object of the present invention is to provide a propulsion assembly for an endoscope and a driving method, in which operability for movement of the endoscope can be equal between proximal and distal directions of the movement.

In order to achieve the above and other objects and advantages of this invention, a propulsion assembly for an endoscope includes a support sleeve for indirect mounting on a tip device of the endoscope. An endless track device is supported on the support sleeve in an endlessly movable manner, for contacting a wall of a body cavity, for propulsion of the tip device relative to the body cavity. A driving mechanism drives the endless track device. At least first and second torque wire devices have proximal and distal end portions, the proximal end portion being rotated by a motor, the distal end portion actuating the driving mechanism. A plurality of first helical windings constitute the first wire device, the first helical windings being so wound as to increase tightness thereof upon moving the endoscope in a distal direction. A plurality of second helical windings constitute the second wire device, the second helical windings being so wound as to increase tightness thereof upon moving the endoscope in a proximal direction.

Furthermore, a first coupling gear is connected to the distal end portion of the first wire device, engaged with the driving mechanism, for outputting torque thereto. A second coupling gear, connected to the distal end portion of the second wire device, meshed with the first coupling gear, for outputting torque thereto.

The first and second helical windings are wound in an equal winding direction.

The first wire device rotates in a first direction and the second wire device rotates in a second direction opposite to the first direction in order to move the endoscope in the distal direction. The first wire device rotates in the second direction and the second wire device rotates in the first direction in order to move the endoscope in the proximal direction.

In another preferred embodiment, while the first wire device is rotated in a first direction, the second wire device is rotated in the first direction.

The second helical windings are wound in a winding direction opposite to a winding direction of the first helical windings.

Furthermore, a first coupling gear is connected to the distal end portion of the first wire device, engaged with the driving mechanism, for outputting torque thereto. A second coupling gear is connected to the distal end portion of the second wire device, engaged with the driving mechanism, for outputting torque thereto.

A total of torsional rigidity of the first and second wire devices upon application of torque for moving the endoscope in the proximal direction to the first and second wire devices is substantially equal to a total of torsional rigidity of the first and second wire devices upon application of torque for moving the endoscope in the distal direction to the first and second wire devices.

Each of the first and second wire devices is single.

The driving mechanism includes a drive sleeve, disposed inside the support sleeve, and rotatable between the support sleeve and the tip device. Spur gear teeth are formed on the drive sleeve, and rotated by the distal end portion of the first and second wire devices.

Furthermore, a clamping mechanism is disposed inside the support sleeve, for maintaining the support sleeve around the tip device.

Furthermore, worm gear teeth are formed on the drive sleeve. A wheel is supported on the support sleeve, rotated by the worm gear teeth, for moving the endless track device.

Also, a driving method for a propulsion assembly includes a support sleeve for mounting on a tip device of an endoscope, an endless track device, supported on the support sleeve in an endlessly movable manner, for contacting a wall of a body cavity, for propulsion of the tip device relative to the body cavity, and a driving mechanism for driving the endless track device. The driving method includes a step of using at least first and second torque wire devices having proximal and distal end portions, the proximal end portion being rotated by a motor, the distal end portion actuating the driving mechanism. The endoscope in a distal direction is moved by rotating the first wire device in a winding direction thereof and by rotating the second wire device in an unwinding direction thereof. The endoscope is moved in a proximal direction by rotating the second wire device in a winding direction thereof and by rotating the first wire device in an unwinding direction thereof.

A first coupling gear is connected to the distal end portion of the first wire device, a second coupling gear is connected to the distal end portion of the second wire device, and the driving mechanism has a third coupling gear.

The second coupling gear is meshed with the first coupling gear, the third coupling gear is meshed with the first coupling gear, and the first and second wire devices rotate in directions opposite to one another.

In another preferred embodiment, the third coupling gear is meshed with each of the first and second coupling gears, and the first and second wire devices rotate in an equal direction opposite to one another.

While the first wire device is rotated in a first direction, the second wire device is rotated in a second direction opposite to the first direction.

In another preferred embodiment, while the first wire device is rotated in a first direction, the second wire device is rotated in the first direction.

Also, an endoscope system is provided, and includes an endoscope having a tip device, and a propulsion assembly. The propulsion assembly includes a support sleeve for mounting on the tip device. An endless track device is supported on the support sleeve in an endlessly movable manner, for contacting a wall of a body cavity, for propulsion of the tip device relative to the body cavity. A driving mechanism drives the endless track device. At least first and second torque wire devices have proximal and distal end portions, the proximal end portion being rotated by a motor, the distal end portion actuating the driving mechanism. A plurality of first helical windings constitute the first wire device, the first helical windings being so wound as to increase tightness thereof upon moving the endoscope in a distal direction. A plurality of second helical windings constitute the second wire device, the second helical windings being so wound as to increase tightness thereof upon moving the endoscope in a proximal direction.

Consequently, operability for movement of the endoscope can be equal between proximal and distal directions of the movement, because of the use of the first and second torque wire devices of which winding directions to increase tightness are different.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope and a propulsion assembly in combination;

FIG. 2 is a perspective view illustrating the propulsion assembly of which an endless track device is developed;

FIG. 3 is an exploded perspective view illustrating the propulsion assembly;

FIG. 4 is an exploded perspective view illustrating a drive sleeve, torque wire devices and motors;

FIG. 4A is a perspective view illustrating the wire devices;

FIG. 5 is a vertical section illustrating the propulsion assembly;

FIG. 6 is an explanatory view in a cross section illustrating the endless track device;

FIG. 7 is an exploded perspective view illustrating another preferred combination of the drive sleeve, the wire devices and the motors;

FIG. 7A is a perspective view illustrating the wire devices.

### DETAILED DESCRIPTION OF THE PREFERRED EMBODIMENT(S) OF THE PRESENT INVENTION

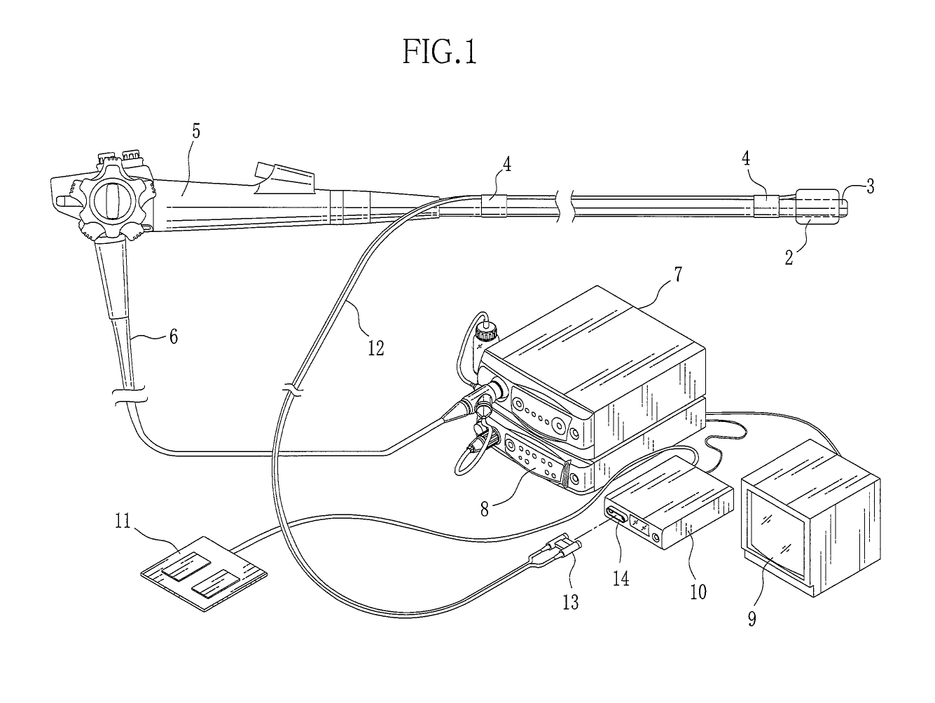

In FIG. 1, a propulsion assembly 2 is for use with an endoscope. The propulsion assembly 2 is fitted around a tip device 3 of the endoscope. The endoscope includes an image sensor, lighting windows, a steering device, an elongated tube, a handle 5, steering wheels and the like. The image sensor is incorporated in the tip device 3, and is a CCD or CMOS image sensor. The lighting windows are formed in the tip device 3 and emit light. The image sensor images an object in a body cavity illuminated with the light from the lighting windows, such an object as a wall of a stomach or intestine of a gastrointestinal tract of a patient. The steering device is disposed at a proximal end of the tip device 3 for steering to enter the tip device 3 in the body cavity to reach the object. The propulsion assembly 2 operates to facilitate the entry of the tip device 3. The steering wheels are disposed on the handle 5, and manually rotated to operate the steering device for bending up and down and to the right and left.

The handle 5 includes a button and an end sleeve. The button is operable to change over the supply and suction of air or water. The end sleeve has an instrument opening where a biopsy forceps or other medical device is advanced. A universal cable 6 extends from the handle 5, and connected to a light source apparatus 7 and a processing apparatus 8. Light from a lamp in the light source apparatus 7 is guided by a light guide fiber extending through the universal cable 6 and the endoscope to the lighting windows. The processing apparatus 8 processes an image signal from the universal cable 6 in the signal processing suitably. A display panel 9 is driven to display the image of the image signal. The processing apparatus 8 discerns the type information of the endoscope for use according to the input information from the endoscope through the universal cable 6. The processing apparatus 8 automatically changes over the control and/or display suitably according to the type information, typically if the control with differences for the types is required in the course of the manipulation, or if the display with differences for the types is required on the display panel 9.

An actuating apparatus 10 or controller is connected with the processing apparatus 8 electrically. The actuating apparatus 10 actuates and controls the propulsion assembly 2. A wire sheath 12 of a dual lumen form extends from a proximal end of the propulsion assembly 2. An adhesive tape 4 or surgical tape positions the wire sheath 12 on the elongated tube of the endoscope at suitable points. The wire sheath 12 extends properly into the body cavity even upon moving the endoscope into the body cavity or during the manipulation.

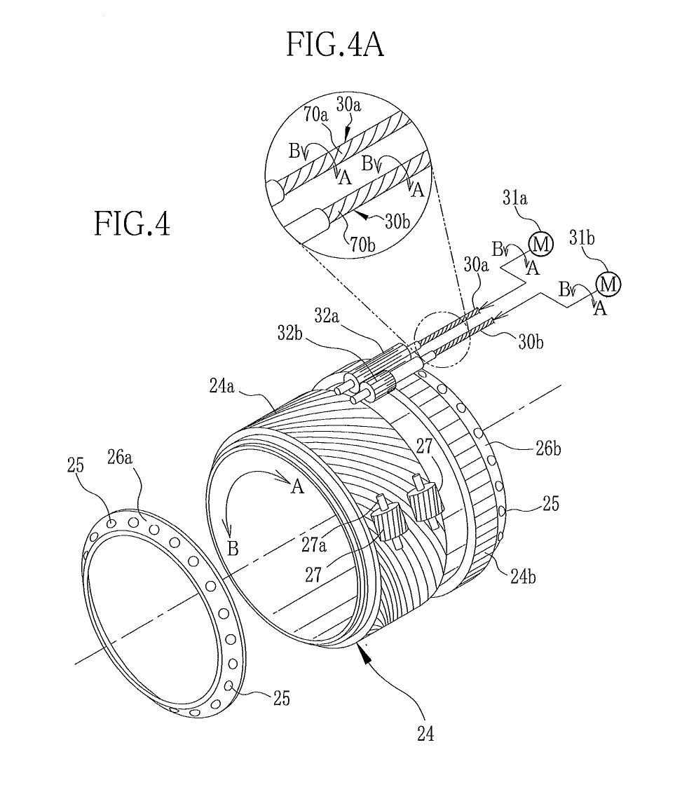

A first torque wire device 30a and a second torque wire device 30b are disposed to extend discretely through the wire sheath 12. See FIGS. 4 and 4A. Distal end portions of the wire devices 30a and 30b are coupled to a driving mechanism (sleeve) of the propulsion assembly 2. The wire devices 30a and 30b are flexible but have high torsional rigidity so that torque applied to their proximal end are transmitted by those to their distal end substantially without attenuation. A key coupling device 13 for plug-in is disposed to the proximal end of the wire devices 30a and 30b. A rotating coupling 14 for plug-in is disposed in the actuating apparatus 10, and connected mechanically with the key coupling device 13. In FIG. 4, a first motor 31a and a second motor 31b are incorporated in the actuating apparatus 10. When the key coupling device 13 is plugged to the rotating coupling 14, the first wire device 30a is ready to rotate with the first motor 31a. The second wire device 30b is ready to rotate with the second motor 31b.

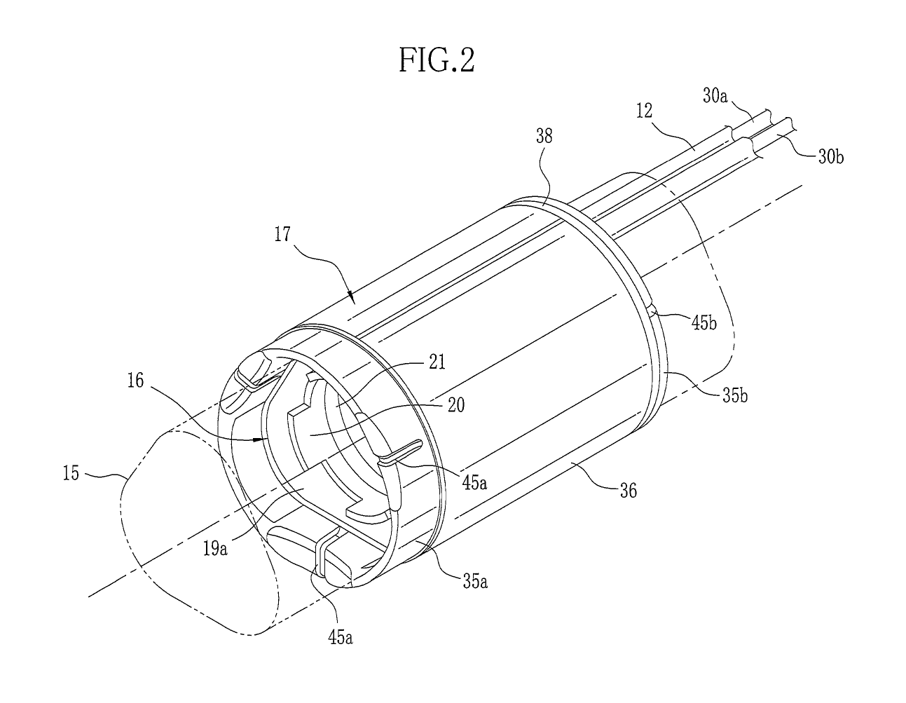

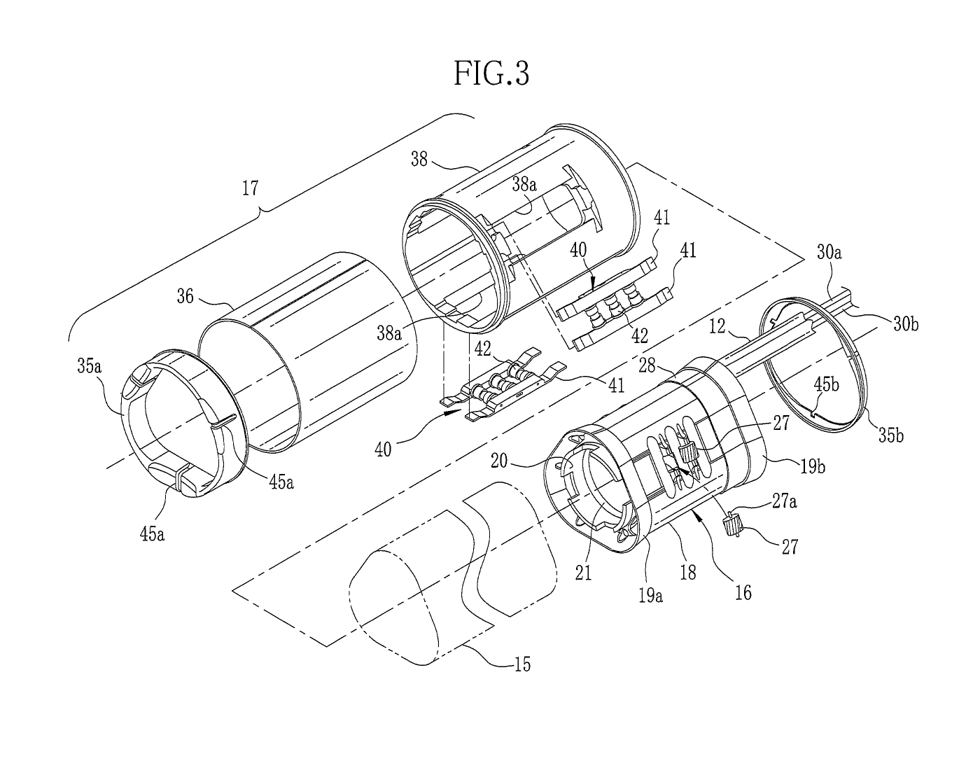

The propulsion assembly 2 is used effectively specially for colonoscopy, because of manipulation for advance and pull in the sigmoid colon or transverse colon. The propulsion assembly 2 is substantially cylindrical. An endless track device 15 or membrane or toroidal device is disposed on the outside of the propulsion assembly 2, is constituted by a flexible sheet of synthetic resin with sufficient rigidity. In FIGS. 2 and 3, the endless track device 15 is depicted in a developed form of a sleeve for understanding. A final form of the endless track device 15 is in a ring shape or toroidal shape after connecting front and rear ends of the sleeve. The endless track device 15 has an annular surface. In FIGS. 2-5, a distal side for protruding the tip device 3 is depicted on the left side. A proximal side near to the handle 5 of the endoscope is depicted on the right side.

In FIGS. 2 and 3, the propulsion assembly 2 includes an inner sleeve unit 16 and an outer sleeve unit 17. The inner sleeve unit 16 is disposed inside the endless track device 15. The outer sleeve unit 17 is disposed around the inner sleeve unit 16. The inner sleeve unit 16 includes a support sleeve 18, a cap ring 28, a distal cover flange 19a for wiping, a proximal cover flange 19b for wiping, a collet sleeve 20, a collet head 21 or C-ring (in a C shape) of a clamping mechanism, and a drive sleeve 24. The support sleeve 18 has a cylindrical inner surface and an outer surface in a shape of a triangular prism. The cap ring 28 is in a triangular shape, and retained to a proximal end of the support sleeve 18 by a screw, press-fit or caulking. The cover flanges 19a and 19b are attached to respectively the distal end of the support sleeve 18 and the proximal end of the cap ring 28. The collet sleeve 20 is helically engaged with a thread formed inside the support sleeve 18, and rotates to move in the axial direction. The collet head 21 is formed from synthetic resin with resiliency, and has a diameter changeable by movement of the collet sleeve in the axial direction. The drive sleeve 24 is a driving mechanism supported inside the support sleeve 18 in a rotatable manner. See FIG. 4.

In FIG. 4, the propulsion assembly 2 includes bearing rings 26a and 26b, each of which is constituted by plural bearing balls arranged annularly. The bearing rings 26a and 26b support ends of the drive sleeve 24 on an inner surface of the support sleeve 18 in a rotatable manner. The cap ring 28 is secured to a proximal end of the support sleeve 18, and prevents the drive sleeve 24 from dropping out. Worm gear teeth 24a or thread, and spur gear teeth 24b are arranged on an outer surface of the drive sleeve 24. Two rotatable worm wheels 27 or helical gears are supported on the support sleeve 18, and meshed with the worm gear teeth 24a through openings in the support sleeve 18. Three pairs of the worm wheels 27 are arranged equiangularly from one another around the drive sleeve 24. When the drive sleeve 24 rotates, the worm wheels 27 rotate around a gear shaft 27a in the same direction simultaneously.

A distal end of the wire sheath 12 is attached to the inside of the proximal end of the cap ring 28 by use of adhesion or thermal welding. Distal ends of the wire devices 30a and 30b protruding from the wire sheath 12 extend to pass through holes in the cap ring 28. First and second coupling gears 32a and 32b or pinions are firmly connected with distal ends of the wire devices 30a and 30b. As illustrated in the drawing, rotational shafts protrude from respectively the coupling gears 32a and 32b as rotational centers. The shafts are received in holes formed in the support sleeve 18, to keep the coupling gears 32a and 32b rotatable. Only the first coupling gear 32a of the first wire device 30a is meshed with the spur gear teeth 24b (third coupling gear) of the drive sleeve 24. The second coupling gear 32b coupled to the second wire device 30b is meshed with the first coupling gear 32a but not with the spur gear teeth 24b. Thus, the drive sleeve 24 is driven by rotation of the first coupling gear 32a in connection with the first wire device 30a. However, the wire devices 30a and 30b are driven by torques generated by respectively the motors 31a and 31b. The second coupling gear 32b is rotated in a direction opposite to that of the first coupling gear 32a. The torque from the second wire device 30b is added to the torque of the first coupling gear 32a, so that the drive sleeve 24 can be rotated with a high torque.

The wire devices 30a and 30b are constituted by helical windings 70a and 70b, for example, helical windings of steel. When the first motor 31a rotates in the direction B in FIG. 4, the first wire device 30a is wound more tightly, to rotate the drive sleeve 24 in the direction A of FIG. 4 to move the tip device 3 in the distal direction. When the second motor 31b rotates in the direction B, the second wire device 30b is wound more tightly, to rotate the drive sleeve 24 in the direction B to move the tip device 3 in the proximal direction. In short, one of the wire devices 30a and 30b is wound tightly at the same time as a remaining one of those is unwound for rotation of the drive sleeve 24.

In general, torsional rigidity of a wire device in a winding direction is higher than torsional rigidity of the same in an unwinding direction. Let one wire device be used for rotating the drive sleeve 24 in two directions. Torsional rigidity of the wire device in rotating the drive sleeve 24 in the direction A is different from torsional rigidity of the wire device in rotating the drive sleeve 24 in the direction B. However, the wire devices 30a and 30b are used according to the invention. The drive sleeve 24 is rotated by rotating one of the wire devices 30a and 30b in a winding direction and a remaining one of the wire devices 30a and 30b in an unwinding direction. Accordingly, the total of the torsional rigidity of the wire devices 30a and 30b is constant irrespective of the rotational directions of the drive sleeve 24.

Each of the cover flanges 19a and 19b includes a flange edge shaped to increase a width in the axial direction. The flange edge receives an inner surface of the endless track device 15 with closeness while the endless track device 15 turns around. The flange edge prevents various materials from pull into the propulsion assembly 2 together with the moving outer surface of the endless track device 15, the materials including foreign material and tissue of a body part.

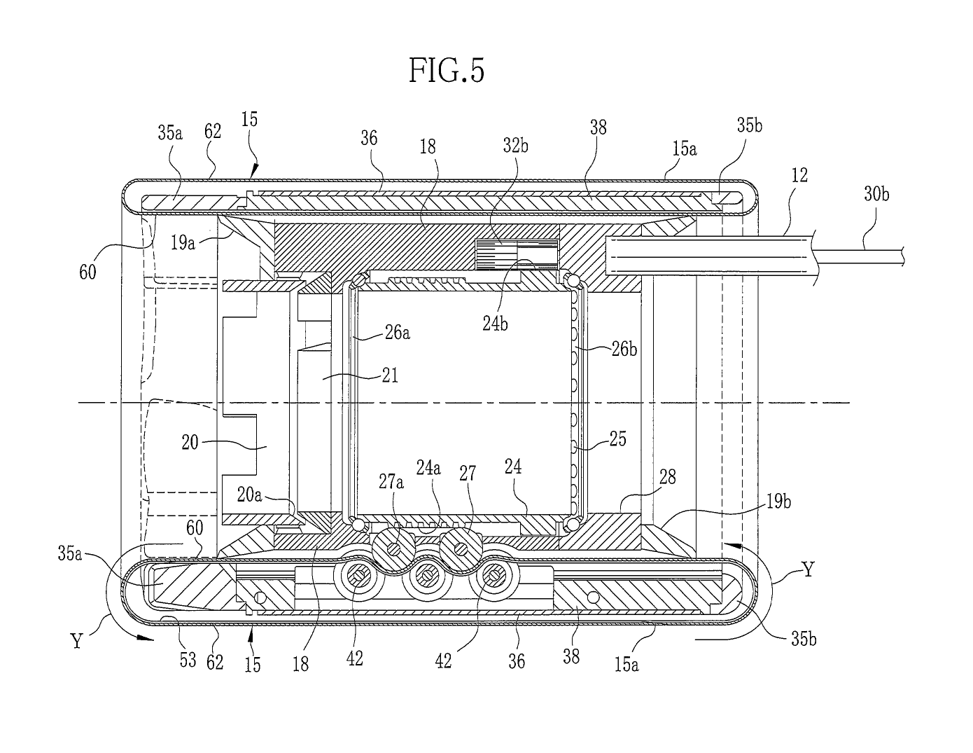

A distal end of the collet sleeve 20 has a pattern of projections and recesses arranged in the circumferential direction. A special tool for the collet sleeve 20 is entered for engagement with the collet sleeve 20 in the proximal direction. The collet sleeve 20 is rotated in a predetermined direction by the tool, and thus shifts in the proximal direction. A tapered end surface 20a of the collet sleeve 20 in FIG. 5 presses the collet head 21, which deforms to decrease the diameter. Accordingly, an inner surface of the collet head 21 is strongly pressed on a peripheral surface of the tip device 3 for firmly fitting the support sleeve 18 thereon.

The outer sleeve unit 17 includes a distal support ring 35a or bumper ring, a cover sheet 36 for shielding, a guide sleeve 38 for supporting rollers, and a proximal support ring 35b or bumper ring, in a sequence in the proximal direction. The outer sleeve unit 17 is combined with the inner sleeve unit 16 and the endless track device 15 according to the steps as follows.

In FIGS. 2 and 3, a sheet material for the endless track device 15 in a developed form is formed in a cylindrical shape. The inner sleeve unit 16 is positioned so that its outer surface is covered inside the cylindrical shape of the sheet material. The inner sleeve unit 16 with the endless track device 15 is entered in the guide sleeve 38. Three holder openings 38a are formed in the guide sleeve 38 to extend in the axial direction, and arranged equiangularly from one another with 120 degrees. Roller mechanisms 40 are mounted in respectively the holder openings 38a.

The roller mechanisms 40 include three idler rollers 42, and a pair of roller supports 41 or frames for supporting the idler rollers 42 in alignment. The roller supports 41 are resilient thin plates of metal, and are fixed to the guide sleeve 38 by fitting their ends in end portions of the holder openings 38a. A center of the roller supports 41 in the longitudinal direction becomes curved to enter an inner space in the guide sleeve 38 through the holder openings 38a. The idler rollers 42 supported by the roller supports 41 press the endless track device 15 toward the worm wheels 27 owing to the curved form of the roller supports 41. As a result, the endless track device 15 is tensioned tightly between the worm wheels 27 and the idler rollers 42. See FIG. 5. There is degree of freedom in one of the idler rollers 42 disposed at the center in relation to the longitudinal direction of the roller supports 41, because the center roller is supported by the opening extending longitudinally. A relative position of the endless track device 15 to two lateral rollers included in the idler rollers 42 is automatically adjusted for supporting the endless track device 15 with the worm wheels 27 in an optimally balanced manner.

The roller mechanisms 40 are fitted in the holder openings 38a fixedly on the guide sleeve 38. The idler rollers 42 project to the inside of the guide sleeve 38 and keep the guide sleeve 38 immovable in the axial direction relative to the inner sleeve unit 16. The endless track device 15 is tensioned while the roller mechanisms 40 are combined with the guide sleeve 38. The support rings 35a and 35b are fixed to respectively the distal and proximal ends of the guide sleeve 38. Three grooves 45a are formed in the distal support ring 35a. Three grooves 45b are formed in the proximal support ring 35b. The grooves 45a and 45b are aligned with the roller mechanisms 40 in the axial direction. The cover sheet 36 tightly covers the outer surface of the guide sleeve 38 together with the roller mechanisms 40.

The sleeve of the endless track device 15 in a developed form is positioned between the inner and outer sleeve units 16 and 17. Those units are combined with one another, before ends of the sleeve of the endless track device 15 are turned over and connected with one another. A joint portion 15a of the endless track device 15 is formed. Note that inclinations can be preferably formed with ends of the sleeve of the endless track device 15, so that the joint portion 15a can have a small thickness without an excessive unevenness of the thickness. In FIG. 5, an assembled structure of the propulsion assembly 2 is schematically illustrated. The endless track device 15 can have an inner space to wrap the outer sleeve unit 17 entirely in the toroidal shape. It is possible to fill the inner space with suitable fluid, such as air, physiological saline water, colloid of synthetic resin, oil, grease, lubricant fluid of various types, and the like.

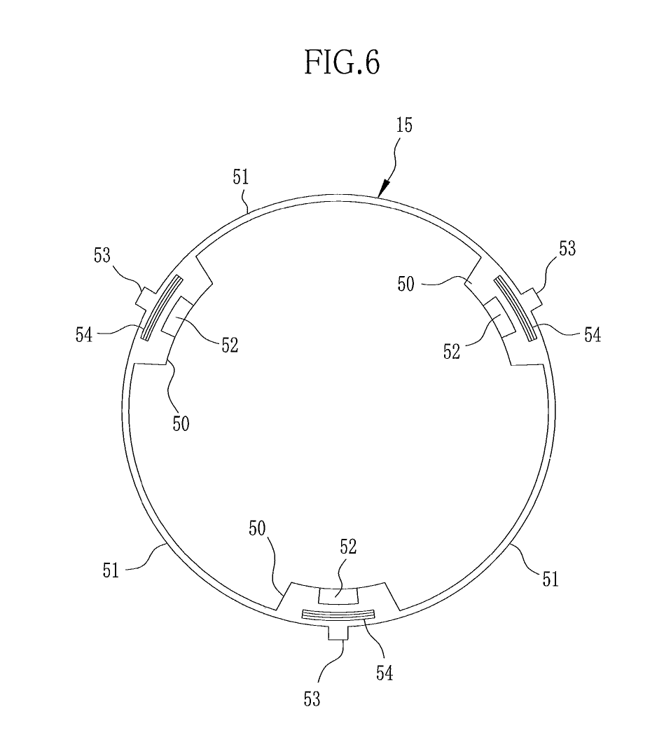

In FIG. 6, a sleeve for forming the endless track device 15 is viewed in a cross section. The endless track device 15 is constituted by a plurality of sheets of polyurethane resin or the like in a multi-layer form. Three reinforcing ridges 50 are formed on an inner sleeve surface of the endless track device 15, arranged equiangularly from one another, and formed in a trapezoidal shape as viewed in section. The reinforcing ridges 50 have a larger thickness than a membrane wall 51, and are constituted by a stack of sheets of a higher number than those in the membrane wall 51. The reinforcing ridges 50 extend longitudinally in the axial direction. Engaging teeth 52 or rack gear teeth are disposed on the surface of the reinforcing ridges 50, and arranged with an inclination for mesh with the worm wheels 27. Alignment ridges 53 are formed on the endless track device 15, extend longitudinally, and are opposite to the reinforcing ridges 50. Also, a mesh sheet 54 of fiber is disposed between the engaging teeth 52 and each of the alignment ridges 53.

The endless track device 15 is used in the toroidal shape in FIG. 5. The three reinforcing ridges 50 are nipped between the worm wheels 27 and the idler rollers 42. The worm wheels 27 are meshed with the engaging teeth 52. Rotation of the worm wheels 27 is transmitted directly to the endless track device 15 by the engaging teeth 52. The endless track device 15 can turn around efficiently in the axial direction. The reinforcing ridges 50 and also the mesh sheet 54 are in the multi-layer form. The engaging teeth 52 in the endless track device 15 can have sufficient mechanical strength even upon receiving driving force directly from the worm wheels 27, because the engaging teeth 52 do not deform or the endless track device 15 does not break. Also, the membrane wall 51 disposed beside the reinforcing ridges 50 is effective in reducing resistance of the endless track device 15 during passage between the inner and outer sleeve units 16 and 17.

Roller grooves are formed in respectively the idler rollers 42 at the center. The alignment ridges 53 disposed opposite to the reinforcing ridges 50 are engaged with the roller grooves when the endless track device 15 moves. Note that the outer sleeve unit 17 can be constructed in an adjustable form for reducing the inner space of the endless track device 15 in a tightly wrapped condition. In this form, the alignment ridges 53 are engaged also with the grooves 45a and 45b of the support rings 35a and 35b. The alignment ridges 53 are effective in stabilizing the path of the movement, as the endless track device 15 can be prevented from shifting in a zigzag manner while moved in the axial direction.

The operation of the above embodiment is described now. In FIG. 1, the propulsion assembly 2 is mounted on the endoscope in a state of protruding a distal end of the tip device 3 partially. A special tool is used for mounting the propulsion assembly 2. The collet sleeve 20 of a clamping mechanism is rotated by the tool in the clockwise direction. The collet sleeve 20 is helically engaged with a female thread formed inside the support sleeve 18 on the distal side. Rotation of the collet sleeve 20 in the clockwise direction shifts the collet sleeve 20 in the inward direction or proximal direction. The tapered end surface 20a presses the collet head 21 or C-ring. A tapered surface on a distal side of the collet head 21 is pressed by the tapered end surface 20a to deform the collet head 21 to decrease its diameter. The tip device 3 is clamped by the collet head 21 inside the support sleeve 18 upon the deformation. The propulsion assembly 2 is fastened to the tip device 3 reliably.

The wire sheath 12 extending from the proximal end of the propulsion assembly 2 is positioned along the outer surface of the steering device and the flexible device of the endoscope. Plural indicia are disposed on the wire sheath 12 equidistantly from one another, and indicate positions of attachment of the adhesive tape 4. The wire sheath 12 is attached to the steering device and the flexible device by use of the adhesive tape 4 according to the indicia. The key coupling device 13 at the proximal end of the wire sheath is plugged to the rotating coupling 14 for connection to the actuating apparatus 10, which is powered. The actuating apparatus 10 checks whether the key coupling device 13 is plugged to the rotating coupling 14 or not upon powering. If it is judged that the plugging is improper or if the plugging is not detected, alarm information is emitted, for example, alarm sound or a visible alarm signal with light. If it is judged that the plugging is proper, a sensor in the rotating coupling 14 reads type information of the propulsion assembly 2 from a signal portion disposed on a bridge portion of the key coupling device 13. According to the type information, the actuating apparatus 10 automatically determines a rotational speed of the wire devices 30a and 30b and a value of a torque limiter, and prevents the wire devices 30a and 30b from operating at too high a speed or torque.

When the power source is turned on, the actuating apparatus 10 receives type information of the endoscope in connection with the processing apparatus 8 in a form of an output signal. The actuating apparatus 10 includes an inner storage medium. The actuating apparatus 10 recognizes the type information of the endoscope for use and type information of the propulsion assembly 2 by referring to table data stored in the storage medium. The table data is data of types of the endoscope and usable types of the propulsion assembly 2 in association with the endoscope types. For example, a shiftable range of the collet head 21 is determined according to the type information of the propulsion assembly 2. An outer diameter of the tip device 3 is determined according to the type information of the endoscope. It is possible promptly to check whether the propulsion assembly 2 can be properly used in connection with the tip device 3 of the endoscope. If it is judged that a combination of the propulsion assembly 2 with the tip device 3 is improper, an alarm signal is generated, for example, alarm sound or visible alarm sign of light with an alarm lamp. Also, operation of the propulsion assembly 2 may be inhibited. Those functions can prevent occurrence of accidents.

When a foot switch 11 in connection with the actuating apparatus 10 is depressed, the motors 31a and 31b in the actuating apparatus 10 rotate to apply torque to the wire devices 30a and 30b. The coupling gears 32a and 32b are caused to rotate, so that the spur gear teeth 24b (third coupling gear) meshed with the first coupling gear 32a are rotated with the drive sleeve 24. The second coupling gear 32b rotates in a direction opposite to that of the first coupling gear 32a. Rotation of the second coupling gear 32b is directly transmitted to the first coupling gear 32a. Thus, the motors 31a and 31b in the actuating apparatus 10 can be utilized to rotate the drive sleeve 24.

To rotate the drive sleeve 24, the wire devices 30a and 30b are used. One of those is rotated in a winding direction. A remaining one of those is rotated in an unwinding direction. A total of the torsional rigidity of the wire devices 30a and 30b is equal irrespective of the directions A and B in which the drive sleeve 24 is rotated. A state of rotating the drive sleeve 24 can be related with operability of manipulating the foot switch 11. There are no problem of higher response to operation of the foot switch 11 upon rotation of the drive sleeve 24 in the direction A, or of lower response to operation of the foot switch 11 upon rotation of the drive sleeve 24 in the direction B. Accordingly, the drive sleeve 24 can rotate smoothly without strange manual touch.

When the worm gear teeth 24a of the drive sleeve 24 rotate, the worm wheels 27 rotate in the same direction about respectively the gear shaft 27a. The endless track device 15 is tensioned between the teeth of the worm wheels 27 and the idler rollers 42 of the roller mechanisms 40. The idler rollers 42 are caused to rotate by the worm wheels 27 to move the endless track device 15 endlessly in the axial direction of the drive sleeve 24. In FIG. 5, the worm wheels 27 rotate in the clockwise direction. The idler rollers 42 rotate in the counterclockwise direction. A return run 60 of the endless track device 15 inside the outer sleeve unit 17 moves from the proximal side to the distal side. A working run 62 of the endless track device 15 outside the outer sleeve unit 17 moves from the distal side to the proximal side. Thus, the endless track device 15 endlessly turns around in the direction Y.

The working run 62 of the endless track device 15 contacts a wall of the large intestine in entry of the endoscope with the propulsion assembly 2 in the gastrointestinal tract. While the endless track device 15 endlessly moves, propulsion force for advancing the tip device 3 is obtained, in other words, force for pressing the wall of the large intestine in the proximal direction is obtained. While the endless track device 15 endlessly moves in a direction backward to the initial direction, propulsion force for returning the tip device 3 is obtained, in other words, force for pressing the wall of the large intestine in the distal direction is obtained. As described heretofore, the endless track device 15 is driven by rotation of the drive sleeve 24, which is controllable with the foot switch 11. The state of manipulating the foot switch 11 is associated with the state of rotating the drive sleeve 24. The drive sleeve 24 can be rotated safely for moving the endoscope back and forth.

During the distal movement of the endoscope, foreign material stuck on the working run 62 of the endless track device 15 may move toward the return run 60 after passing the proximal end of the outer sleeve unit 17. However, the flange edge of the proximal cover flange 19b is positioned very close to the endless track device 15 and prevents the foreign material from internal jamming. Also, the proximal cover flange 19b prevents tissue of a body part from internal jamming together with the endless track device 15. Note that during the proximal movement of the endoscope, the flange edge of the distal cover flange 19a operates in the same manner for protection.

If the operator wishes to remove the propulsion assembly 2 from the tip device 3, the collet sleeve 20 is rotated in the counterclockwise direction by use of the tool. The collet sleeve 20 shifts in an outward direction by rotating, and releases the collet head 21 from being pressed. The collet head 21 is enlarged by its resiliency to separate its inner surface from an outer surface of the tip device 3. The propulsion assembly 2 can be removed from the endoscope easily.

According to the invention, helical windings of one of the first and second wire devices are wound to increase their tightness in the course of the propulsion. Helical windings of a remaining one of the first and second wire devices are loosened at the same time. The total of the torsional rigidity of the first and second wire devices is set equal between the proximal movement and distal movement of the endoscope. It is possible to modify specific details of the structure according to the invention. In the above embodiments, the number of each of the first and second wire devices is one. However, two or more wire devices can constitute each of the first and second wire devices. The number of the first wire device may be different from that of the second wire device.

In each of the first and second wire devices, a structure of combining the plural helical windings can be according to well-known types. For example, the first and second wire devices can be a nested type in which helical windings of plural diameters are combined, a type of a multiple helix in which helical windings are combined with a difference in the axial direction, a combination of the nested type and the multiple helix, and the like.

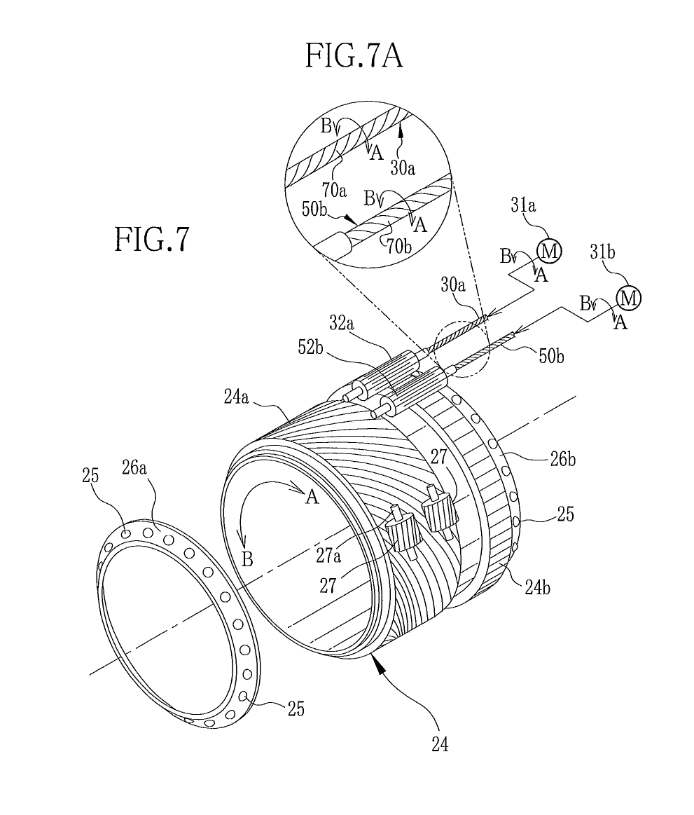

In the above embodiment, the second wire device 30b is indirectly connected with the drive sleeve 24, as the first wire device 30a transmits torque of the second wire device 30b to the drive sleeve 24. However, the invention is not limited to this feature. In FIGS. 7 and 7A, there is a second torque wire device 50b. A second coupling gear 52b is disposed at an end of the second wire device 50b in the same form as the first wire device 30a. The second coupling gear 52b is directly meshed with the spur gear teeth 24b (third coupling gear) of the drive sleeve 24. The second wire device 50b can apply torque directly to the drive sleeve 24 without utilizing the first wire device 30a. In the embodiment, the drive sleeve 24 rotates in the direction B when the second wire device 50b rotates in the direction A, and rotates in the direction A when the second wire device 50b rotates in the direction B. This is a feature distinct from the first embodiment. The second wire device 50b of the present embodiment includes the plural helical windings 70b wound in a direction opposite to the winding direction of those in the second wire device 30b of FIGS. 4 and 4A. A total of the torsional rigidity of the wire devices 30a and 50b is constant irrespective of the rotational directions of the drive sleeve 24. Elements similar to those of the above embodiments are designated with identical reference numerals in FIGS. 7 and 7A.

In the above embodiments, the inner sleeve unit 16 is triangular. However, the inner sleeve unit 16 can be shaped in a cylindrical form, a form of a polygonal prism, and the like. In the above embodiments, the outer sleeve unit 17 is cylindrical. However, the outer sleeve unit 17 can be shaped in a form of a triangular prism, polygonal prism and the like.

In the above embodiments, the endless track device is in a toroidal shape. However, an endless track device of the invention may include a plurality of endless belts arranged in a circumferential direction of the outer sleeve unit and extending in the axial direction.

In the above embodiment, the endless track device 15 is moved endlessly by the combination of the worm gear teeth 24a and the worm wheels 27 in the drive sleeve 24. However, it is possible to engage the worm gear teeth 24a with the endless track device 15 without the worm wheels 27 to drive the endless track device 15 directly.

In the above embodiments, the drive sleeve 24 is positioned as an innermost sleeve in the propulsion assembly, and rotates between the tip device 3 of the endoscope and the support sleeve 18. The collet sleeve 20 and the collet head 21 fit the propulsion assembly around the tip device 3 by clamping on the distal side from the drive sleeve 24. However, various clamping structures of known forms can be used for fixedly fitting the propulsion assembly around the tip device 3. For example, a shaft sleeve may be disposed between the drive sleeve 24 and the tip device 3, for fixedly fitting the propulsion assembly around the tip device 3.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly for an endoscope comprising:
a support sleeve for indirect mounting on a tip device of said endoscope;
an endless track device, supported on said support sleeve in an endlessly movable manner, for contacting a wall of a body cavity, for propulsion of said tip device relative to said body cavity;
a driving mechanism for driving said endless track device;
at least first and second torque wire devices, having proximal and distal end portions, said proximal end portion being rotated by a motor, said distal end portion actuating said driving mechanism;
a plurality of first helical windings for constituting said first wire device, said first helical windings being so wound as to increase tightness thereof upon moving said endoscope in a distal direction;
a plurality of second helical windings for constituting said second wire device, said second helical windings being so wound as to increase tightness thereof upon moving said endoscope in a proximal direction.

2. A propulsion assembly as defined in claim 1, further comprising:
a first coupling gear, connected to said distal end portion of said first wire device, engaged with said driving mechanism, for outputting torque thereto;
a second coupling gear, connected to said distal end portion of said second wire device, meshed with said first coupling gear, for outputting torque thereto.

3. A propulsion assembly as defined in claim 2, wherein said first and second helical windings are wound in an equal winding direction.

4. A propulsion assembly as defined in claim 3, wherein said first wire device rotates in a first direction and said second wire device rotates in a second direction opposite to said first direction in order to move said endoscope in said distal direction;
said first wire device rotates in said second direction and said second wire device rotates in said first direction in order to move said endoscope in said proximal direction.

5. A propulsion assembly as defined in claim 1, further comprising:
a first coupling gear, connected to said distal end portion of said first wire device, engaged with said driving mechanism, for outputting torque thereto;
a second coupling gear, connected to said distal end portion of said second wire device, engaged with said driving mechanism, for outputting torque thereto.

6. A propulsion assembly as defined in claim 5, wherein said second helical windings are wound in a winding direction opposite to a winding direction of said first helical windings.

7. A propulsion assembly as defined in claim 6, wherein said first and second wire devices rotate in a first direction in order to move said endoscope in said distal direction, and rotate in a second direction opposite to said first direction in order to move said endoscope in said proximal direction.

8. A propulsion assembly as defined in claim 1, wherein a total of torsional rigidity of said first and second wire devices upon application of torque for moving said endoscope in said proximal direction to said first and second wire devices is substantially equal to a total of torsional rigidity of said first and second wire devices upon application of torque for moving said endoscope in said distal direction to said first and second wire devices.

9. A propulsion assembly as defined in claim 8, wherein each of said first and second wire devices is single.

10. A propulsion assembly as defined in claim 1, wherein said driving mechanism includes:
a drive sleeve, disposed inside said support sleeve, and rotatable between said support sleeve and said tip device;
spur gear teeth, formed on said drive sleeve, and rotated by said distal end portion of said first and second wire devices.

11. A propulsion assembly as defined in claim 10, further comprising a clamping mechanism, disposed inside said support sleeve, for maintaining said support sleeve around said tip device.

12. A propulsion assembly as defined in claim 10, further comprising:
worm gear teeth formed on said drive sleeve;
a wheel, supported on said support sleeve, rotated by said worm gear teeth, for moving said endless track device.

13. A driving method for a propulsion assembly including a support sleeve for indirect mounting on a tip device of an endoscope, an endless track device, supported on said support sleeve in an endlessly movable manner, for contacting a wall of a body cavity, for propulsion of said tip device relative to said body cavity, and a driving mechanism for driving said endless track device, said driving method comprising steps of:
using at least first and second torque wire devices having proximal and distal end portions, said proximal end portion being rotated by a motor, said distal end portion actuating said driving mechanism;
moving said endoscope in a distal direction by rotating said first wire device in a winding direction thereof and by rotating said second wire device in an unwinding direction thereof;
moving said endoscope in a proximal direction by rotating said second wire device in a winding direction thereof and by rotating said first wire device in an unwinding direction thereof.

14. A driving method as defined in claim 13, wherein a first coupling gear is connected to said distal end portion of said first wire device, a second coupling gear is connected to said distal end portion of said second wire device, and said driving mechanism has a third coupling gear.

15. A driving method as defined in claim 14, wherein said second coupling gear is meshed with said first coupling gear, said third coupling gear is meshed with said first coupling gear, and said first and second wire devices rotate in directions opposite to one another.

16. A driving method as defined in claim 14, wherein said third coupling gear is meshed with each of said first and second coupling gears, and said first and second wire devices rotate in an equal direction opposite to one another.
